(19) United States
(12) Patent Application Publication
(10) Pub. No.: US 2024/0382397 A1
(43) Pub. Date: Nov. 21, 2024

(54) POWDER COSMETIC

(71) Applicant: SHISEIDO COMPANY, LTD., Chuo-ku, Tokyo (JP)

(72) Inventors: Yui YAMAGUCHI, Tokyo (JP); Tatsuya YONEMIZU, Tokyo (JP)

(73) Assignee: SHISEIDO COMPANY, LTD., Chuo-ku, Tokyo (JP)

(21) Appl. No.: 18/692,081

(22) PCT Filed: Oct. 13, 2022
(86) PCT No.: PCT/JP2022/038206

§ 371 Date: Mar. 14, 2024

(30) Foreign Application Priority Data
Oct. 14, 2021 (JP) 2021-168820

(51) Int. Cl. A61K 8/29; A61K 8/19; A61K 8/25; A61K 8/26; A61K 8/27; A61Q 1/02

(52) U.S. Cl. CPC A61K 8/29; A61K 8/19; A61K 8/25; A61K 8/26; A61K 8/27; A61Q 1/02; A61K 2800/43

(57) ABSTRACT

The present invention provides a powder cosmetic which is highly effective in protection against ultraviolet light because of the inclusion of a powder of a metal oxide, e.g., titanium dioxide, as an ultraviolet-scattering agent and which has excellent texture to give no frictional feeling when applied. This powder cosmetic comprises (a) a metal oxide powder consisting of at least one selected from titanium dioxide, zinc oxide, and cerium oxide; and (b) an extender pigment, wherein each of the (a) metal oxide powder and the (b) extender pigment is a powder surface-treated with metal soap, and wherein the amount of the (a) metal oxide powder is 10% by mass or more with respect to the total amount of the cosmetic.

### TECHNICAL FIELD

The present invention relates to a powder cosmetic. More specifically, the present invention relates to a powder cosmetic that has a high UV protection ability based on a UV scattering agent such as titanium dioxide and further has excellent texture free from a squeaking feeling at the time of application.

### BACKGROUND ART

Improvement of the UV protection ability of powder cosmetics such as face powders requires increasing the amounts of UV absorbing agents or UV scattering agents. However, large amounts of UV absorbing agents, which are mostly oily substances, may induce aggregation of the powder and impair the uniformity and texture of the cosmetics.

On the other hand, increasing amount of UV scattering agents such as titanium dioxide, zinc oxide or cerium oxide causes a squeaking feeling. Thus, it has been difficult to obtain a powder cosmetic having an improved UV protection ability to achieve high sun protection factor (SPF) while maintaining the suitable texture of the powder cosmetic.

Patent Document 1 describes a powder cosmetic containing (a) an amino-modified-silicone-treated powder and (b) a plate-like zinc oxide having an average particle size of 30 nm or more and less than 1000 nm and an aspect ratio of 3.0 or more at a predetermined mass ratio, and states that the powder cosmetic causes no squeaking feeling and has an excellent UV blocking effect.

Patent Document 2 describes a solid powder cosmetic containing: (A) a composite powder having an average particle size of 0.5 to 200 μm comprising a plate-like base powder on which (a) a UV scattering agent having an average particle size of 0.1 to 1 μm, (b) iron oxide, and (c) an amino acid or a derivative thereof are coated; and (B) a hydrogenated castor oil or an ester thereof, and states that the powder cosmetic causes no squeaking feeling at the time of application and can finish the skin as a bare skin with dullness-free.

However, a powder cosmetic having an improved UV protection ability based on a metal oxide powder such as titanium dioxide as a UV scattering agent and is further free from a squeaking feeling has not yet been obtained. Since it has nowadays become common to wear masks to prevent the spread of infectious diseases such as COVID-19, the number of consumers demanding high SPF in powder cosmetics that can be easily applied has increased.

### CITATION LIST

### Patent Document

Patent Document 1: JP-B 6231411

Patent Document 2: JP-A 2017-81858

### SUMMARY OF INVENTION

### Technical Problem

An object of the present invention is to provide a powder cosmetic that has a high UV protection ability with a large amount of metal oxide powder such as titanium dioxide as a UV scattering agent and further has excellent texture free from a squeaking feeling at the time of application.

### Solution to Problem

The present inventors have intensive studies to solve the above problems. As a result, the present inventors have found that surface-treating both of a metal oxide powder such as titanium dioxide, which is a UV scattering agent, and an extender pigment, which is contained to constitute a powder cosmetic containing the metal oxide powder, with metal soap can achieve excellent texture free from squeakiness even when a large amount of metal oxide powder such as titanium dioxide is contained, and have completed the present invention.

That is, the present invention provides a powder cosmetic, comprising:
(a) a metal oxide powder consisting of at least one selected from titanium dioxide, zinc oxide, and cerium oxide; and
(b) an extender pigment,
wherein each of the (a) metal oxide powder and the (b) extender pigment is a powder surface-treated with metal soap, and
wherein the amount of the (a) metal oxide powder is 10% by mass or more with respect to the total amount of the cosmetic.

### Advantageous Effects of Invention

The powder cosmetic of the present invention has a high UV protection effect based on a large amount of 10% by mass or more of a metal oxide powder such as titanium dioxide, which is a UV scattering agent, while having excellent texture free from a squeaking feeling. In addition, the powder cosmetic of the present invention has an unexpected effect of further improving the UV protection effect (hereinafter also referred to as “boosting effect”) by unifying the surface treatments of the UV scattering agent and the extender pigment into a metal soap treatment.

### BRIEF DESCRIPTION OF DRAWINGS

FIG. 1 A graph showing the absorbance in the UV region of compositions containing a metal soap-treated titanium dioxide with a metal soap-treated talc (solid line) or a silicone-treated talc (dashed line).

FIG. 2 A graph plotting the integral value of the absorbance in the UV region of compositions prepared with combinations of a metal soap-treated titanium dioxide and a metal soap-treated mica (⋅), a metal soap-treated titanium dioxide and a silicone-treated mica (▾), or a silicone-treated titanium dioxide and a silicone-treated mica (▪), with respect to the amount of the titanium dioxide contained.

FIG. 3 A graph showing the absorbance in the UV region of the cosmetics of Example 4 and Comparative Example 2.

### DESCRIPTION OF EMBODIMENTS

The powder cosmetic of the present invention (hereinafter also referred to simply as “cosmetic”) is characterized in that the cosmetic comprises (a) a metal oxide powder: and (b) an extender pigment, and each of the (a) metal oxide powder and the (b) extender pigment is surface-treated with metal soap.

### (a) Metal Oxide Powder

The (a) metal oxide powder (hereinafter also referred to as “component (a)”) in the cosmetic of the present invention is a fine particulate metal oxide powder consisting of at least one selected from titanium dioxide (or TiO₂), zinc oxide (or ZnO), and cerium oxide (or CeO₂), and used as a “UV scattering agent” in cosmetics. The metal oxide powder (component a) typically has an average particle size of 200 nm or less, preferably 100 nm or less. In the present specification, a metal oxide powder having an average particle size of 200 nm or less is referred to as a “metal oxide powder (titanium dioxide, zinc oxide or cerium oxide)” or “UV scattering agent”, and a metal oxide powder having an average particle size of more than 200 nm is distinctively referred to as a “pigment grade metal oxide powder”. In the present invention, titanium dioxide is particularly preferably used among the metal oxide powders.

The metal oxide powder (component (a)) according to the present invention is not particularly limited as long as it has been conventionally used in cosmetics and the like. For example, a metal oxide powder in the form of fine particles or ultra-fine particles with an average primary particle size of 100 nm or less, 50 nm or less, 30 nm or less, 20 nm or less, or 10 nm or less is preferably used. In particular, the smaller the average primary particle size, the more significant the synergistic improvement of the UV protection effect by co-formulating with the extender pigment described later. The lower limit of the average primary particle size of the metal oxide powder (component (a)) is not particularly limited, but is usually 1 nm or more, preferably 5 nm or more.

The average primary particle size used herein means the arithmetic average diameter of the primary particles of the powder as measured by commonly used methods. As used herein, the Feret diameter of the powder particles measured by the microscopy method is taken as the primary particle size of the particles, and the arithmetic average value calculated from the primary particle size distribution (particle size distribution) determined for the plurality of particles is taken as the average primary particle size (or average particle size). The Feret diameter is the distance between two parallel lines when the projected image of the particle is sandwiched by the two parallel lines in a certain direction.

The particle form of the metal oxide powder such as titanium dioxide is not particularly limited, and examples thereof include spherical, elliptical, and crushed forms.

The amount of the (a) metal oxide powder in the cosmetic of the present invention is preferably 10% by mass or more, further preferably 15% by mass or more, with respect to the total amount of the cosmetic, as a metal oxide powder containing a surface treatment agent or as a pure amount of the metal oxide powder (a portion excluding the surface treatment agent). When the amount of the (a) metal oxide powder is less than 10% by mass, the UV protection effect may not be sufficient. The upper limit of the amount of the (a) metal oxide powder is not particularly limited, but is usually 40% by mass or less, preferably 30% by mass or less.

### (b) Extender Pigment

The “extender pigment” in cosmetics is an inorganic pigment used to maintain the dosage form of powder cosmetics. Specific examples thereof include powders of talc, mica, sericite, kaolin, calcium carbonate, magnesium carbonate, silisic anhydrous, aluminum oxide, barium sulfate, and the like.

The amount of the (b) extender pigment in the cosmetic of the present invention is not particularly limited, but is usually 30 to 60% by mass, preferably 35to 50% by mass with respect to the total amount of the cosmetic, as an extender pigment containing a surface treatment agent or as a pure amount of the extender pigment (a portion excluding the surface treatment agent).

The cosmetic of the present invention is characterized in that each of the (a) metal oxide powder and the (b) extender pigment is surface-treated with “metal soap”. That is, the “component (a)” of the present invention can be reworded as the “(a) metal oxide powder surface-treated with metal soap” and the “component (b)” can be reworded as the “(b) extender pigment surface-treated with metal soap”.

The “metal soap” is a salt of a saturated or unsaturated higher fatty acid with metal (other than alkali metal). The higher fatty acid that constitutes the metal soap is not particularly limited, but examples thereof include saturated and/or unsaturated higher fatty acids having 8 to 24 carbon atoms, particularly 12 to 18 carbon atoms, such as stearic acid, isostearic acid, myristic acid, and lauric acid. The metal constituting the metal soap is preferably a salt of aluminum, calcium, magnesium, zinc, or the like.

Specific examples of the metal soap preferably used as the surface treatment agent for the components (a) and component (b) of the present invention include aluminum stearate, aluminum myristate, magnesium myristate, zinc stearate, calcium stearate, zinc myristate, zinc oleate, magnesium isostearate, magnesium stearate, aluminum distearate, aluminum oleate, aluminum palmitate, aluminum laurate, and aluminum dimyristate.

The “powder (metal oxide powder or extender pigment) treated with metal soap” in the present invention is a powder in which a powder to be a base (mother core) is surface-treated with a treatment agent containing a component constituting the metal soap.

The surface treatment with metal soap may be performed by a method of surface-treating with a pre-formed metal soap. For example, the surface treatment can be performed by dissolving metal soap in a volatile solvent such as isoparaffin, isopropyl alcohol, or the like, mixing it with a base powder, and then volatilizing the volatile solvent. The surface treatment can also be performed by simply mixing a base powder with metal soap. Alternatively, the surface treatment may be performed by a method of compounding a higher fatty acid (e.g., stearic acid) that constitutes metal soap with a hydroxide of metal (e.g., aluminum). The surface treatment method is not particularly limited, but examples thereof include a wet process using a solvent, a gas phase process, and a mechanochemical process.

The amount of the metal soap adhered to the surface of the powder of the (a) metal oxide powder or the (b) extender pigment by the surface treatment is not particularly limited, but is preferably about 10 to 40% by mass with respect to the powder to be a base (mother core).

The kinds (the combination of the higher fatty acid and the metal) of the metal soap that adheres to the surfaces of the powders by the surface treatments of the (a) metal oxide powder and the (b) extender pigment are not particularly limited. The metal soap adhered to the surface of the (a) metal oxide powder and the metal soap adhered to the surface of the (b) extender pigment may be the same or different. In the present invention, it is preferable to have a combination of a metal oxide (preferably titanium dioxide) surface-treated with metal soap containing aluminum stearate and an extender pigment (preferably talc or mica) surface-treated with metal soap containing calcium stearate.

The cosmetic of the present invention is a powder cosmetic comprising the (a) metal oxide powder treated with metal soap and the (b) extender pigment surface-treated with metal soap, and the powder cosmetic may further comprise other optional components conventionally used in powder cosmetics to the extent that the effects of the present invention are not impaired.

When the powder cosmetic of the present invention comprises (c) silica (silisic anhydrous; also referred to as “component (c)”), an effect that further improves the UV protection effect (a boosting effect) can be obtained.

The silica, as the component (c), is preferably spherical silica. The term “spherical” herein does not mean that the shape is always a true sphere and means that the shape in which the ratio of major/minor diameters of particles is 1.5 or less, preferably 1.2 or less.

The silica (component (c)) may be non-porous or porous. The silica (component (c)) is a spherical powder having, but not particularly limited to, an average particle size of 1 to 30 μm, preferably 1 to 20 μm, more preferably 2 to 15 μm. The average particle size of silica is the arithmetic average value of particle size measured according to the laser diffraction and scattering method.

In particular, it is preferred to use spherical silica with a specific surface area of 160 m²/g or more, preferably 300 m²/g or more, more preferably 500 m²/g or more, to obtain a good boosting effect.

The specific surface area of silica can be calculated by measuring the amount of nitrogen adsorbed to the powder at 77 K and analyzing it by the BET method.

It should be noted that the (c) silica is an untreated silica that has not been surface-treated, or a silica that has been surface-treated with a treatment agent other than metal soap. In other words, the (c) silica does not include silica surface-treated with metal soap, i.e., the (c) silica does not include powder that falls under the component (b).

Examples of the other optional components include a powder component other than the (a) metal oxide powder surface-treated with metal soap, the (b) extender pigment surface-treated with metal soap and the (c) silica: an oily component: and an aqueous component.

Examples of the powder component other than the (a) metal oxide powder surface-treated with metal soap, the (b) extender pigment surface-treated with metal soap and the (c) silica include a color pigment such as iron oxide: a white pigment such as pigment-grade titanium dioxide or pigment-grade zinc oxide: a pearlescent pigment: and a spherical powder of boron nitride or the like.

These optional powder components may or may not be surface-treated.

Examples of the oily component include an oil such as a hydrocarbon oil, an oil and fat, a higher alcohol, a wax, a hardening oil, an ester oil, a fatty acid, a silicone oil, a fluorine-based oil, a lanolin derivative, an oily gelling agent, and an oily UV absorbing agent.

It should be noted that since an oil may induce aggregation of the powder, the amount of oil in the cosmetic of the present invention is preferably 10% by mass or less, more preferably 5% by mass or less, further preferably 3% by mass or less, most preferably 2% by mass or less, with respect to the total amount of the cosmetic.

In the powder cosmetic of the present invention, the amount of oil contained is suppressed, so that the amount of the oily UV absorbing agent is also small (for example, 2% by mass or less), but since the UV protection effect based on the UV scattering agent is improved (boosted), high SPF can be achieved. Thus, it is also possible to make a powder cosmetic that does not contain an oily UV absorbing agent (so-called “non-chemical” powder cosmetic).

Examples of the aqueous component can include water and water-affinity components, for example, a lower alcohol such as ethanol, a polyol such as glycerin, and a water-soluble polymer.

The cosmetic of the present invention is a powder cosmetic excellent in UV protection effect and in texture, and can be provided in the form of loose powder which is not subjected compression-molding or pressed powder which is subjected compression-molding. The form of products provided is not limited, but the cosmetic of the present invention is particularly suitable for products such as foundation, eye shadow, blush, and sunscreen.

### EXAMPLES

Hereinafter, the present invention is described in further detail by way of Examples and the like. However, the following Examples and the like do not limit in any way the scope of the present invention. Unless specified, the amount represents % by mass with respect to the total amount of the cosmetic.

### Test Example 1

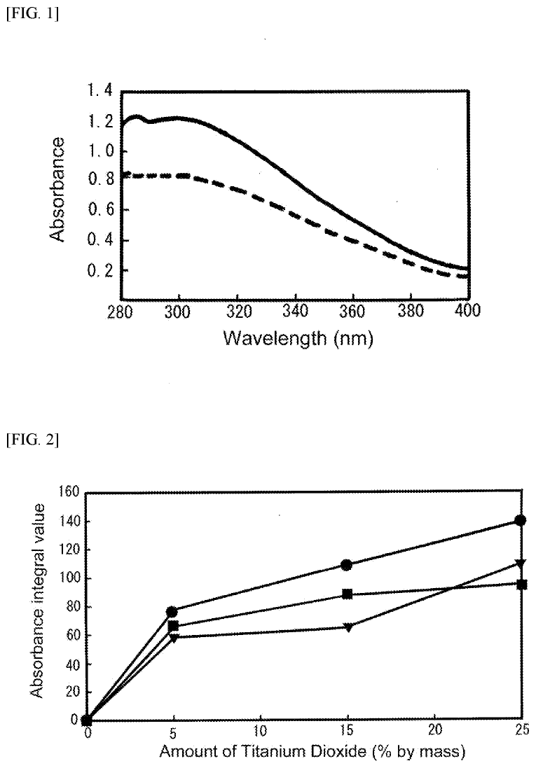

Compositions of samples A1 and A2 were prepared with the formulations listed in Table 1 below. 10 mg of each sample was applied to a PMMA plate and subjected to a measurement of absorbance to light in the UV wavelength region of 280 to 400 nm. The measurement results are shown in FIG. 1.

TABLE 1

| | Sample A1 | Sample A2 |
|---|---|---|
| Metal soap-treated titanium dioxide (*1) | 15 | 15 |
| Metal soap-treated talc (*2) | Remaining | — |
| | amount | |
| Silicone-treated talc (*3) | — | Remaining |
| | | amount |
| Total | 100 | 100 |

(*1) Aluminum stearate-treated titanium dioxide (average particle size =10 nm or less)

(*2) Calcium stearate-treated talc

(*3) Dimethicone-treated talc

As shown in FIG. 1, the absorbance in the UV wavelength region of sample A1 (solid line) in which 15% by mass of metal soap-treated titanium dioxide was dispersed in metal soap-treated talc was improved compared to that of sample A2(dashed line) in which the same amount of metal soap-treated titanium dioxide was dispersed in a silicone-treated talc.

It should be noted that the absorbance curve of a sample in which the same amount of metal soap-treated titanium dioxide as in samples A1 and A2 was applied to the above-described PMMA plate without the extender pigment (talc) and the same measurement was performed was equivalent to the curve of sample A2 (dashed line). In other words, it was confirmed that an improvement effect (a boosting effect) of UV protection ability based on titanium dioxide was obtained by unifying the surface treatments of titanium dioxide and the extender pigment into a metal soap treatment.

### Test Example 2

Compositions of samples B1 to B3 of the formulations listed in Table 2 below were prepared. “X” in Table 2 represents the amount of titanium dioxide (% by mass), and this amount (X) was changed to 5, 15, and 25% by mass. 10 mg of each sample was applied to a PMMA plate, and subjected to a measurement of absorbance to light in the UV wavelength region of 280 to 400 nm. The absorbance curve generated in the measured wavelength region was integrated to give an absorbance integral value, and the obtained results (absorbance integral value) were plotted to give the graph in FIG. 2.

TABLE 2

| | Sample B1 | Sample B2 | Sample B3 |
|---|---|---|---|
| Metal soap-treated titanium dioxide | X | X | — |
| (*1) | | | |
| Silicone-treated titanium dioxide (*4) | — | — | X |
| Metal soap-treated mica (*5) | Remaining | — | — |
| | amount | | |
| Silicone-treated mica (*6) | — | Remaining | Remaining |
| | | amount | amount |
| Total | 100 | 100 | 100 |

(*4) Silicone-treated titanium dioxide

(*5) Magnesium stearate-treated mica

(*6) Hydrogen dimethicone-treated mica

As shown in FIG. 2, sample B1 (⋅) in which metal soap-treated titanium dioxide was dispersed in metal soap-treated mica continues to show an increasing trend in absorbance integral values even when the amount of titanium dioxide is increased to 25% by mass. In contrast, sample B2 (▾) in which metal soap-treated titanium dioxide was dispersed in silicone-treated mica, and sample B3 (▪) in which the surface treatments of the extender pigment (mica) and titanium dioxide were unified into a silicone treatment showed that the absolute value of the absorbance integral value was lower than that of sample B1 and the increase in the absorbance integral value along with the increase in the amount of titanium dioxide tended to reach a plateau.

### Examples and Comparative Examples

Powder cosmetics of the formulation listed in Table 3 were prepared by the conventional method, and the cosmetics of each example were evaluated for UV protection ability and texture (absence of squeakiness). These results are also shown in Table 3.

In the same manner as Test Examples 1 and 2 described above, 10 mg of the cosmetic of each example was applied to a PMMA plate and subjected to a measurement of absorbance to light in the UV wavelength region of 280 to 400 nm, and then the absorbance in the UV wavelength region was integrated.

“Comparative Example 1” and “Example 1” in Table 3 correspond to “Sample A2” and “Sample A1” in Table 1, respectively. Accordingly, the absorbance curves obtained for cosmetics of these examples are as shown in FIG. 1. Here, the integral value of the absorbance curve obtained from the cosmetic of “Comparative Example 1” was defined as the “reference integral value” and each integral value of the absorbance curves obtained from the cosmetics of Examples 1 to 3 was defined as the “measured integral value”, and the value calculated by the following expression was defined as “boost ratio” and used as an evaluation index of the UV protection ability.

$$Boost ratio = ( measured integral value / reference integral value ) \times 100$$

That is, a cosmetic having a “boost ratio” of more than 100 means that the cosmetic has an improved UV protection ability (has a boosting effect) by unifying the surface treatments of titanium dioxide and the extender pigments into a metal soap treatment, compared to the case where the surface treatments of the two are different. It also shows that the higher the value of the boost ratio is, the greater the boosting effect is.

The texture (absence of squeakiness) was evaluated according to the following criteria by sensory testing of each cosmetic used by a specialized panel of 3 people.
A: There is almost no or little squeaking feeling.
B: There is an obvious squeaking feeling.

TABLE 3

| | Comparative | | | |
|---|---|---|---|---|
| | Example 1 | Example 1 | Example 2 | Example 3 |
| Metal soap-treated talc (*2) | — | Remaining | Remaining | Remaining |
| | | amount | amount | amount |
| Silicone-treated talc (*3) | Remaining | — | — | — |
| | amount | | | |
| Metal soap-treated titanium | 15 | 15 | — | — |
| dioxide (*1) | | | | |
| Metal soap-treated titanium | — | — | 15 | — |
| dioxide (*7) | | | | |
| Metal soap-treated titanium | — | — | — | 15 |
| dioxide (*8) | | | | |
| Total | 100 | 100 | 100 | 100 |
| UV protection ability (boost ratio) | Reference | 142 | 104 | 112 |
| Texture (absence of | B | A | A | A |
| squeakiness) | | | | |

(*7) Aluminum stearate-treated titanium dioxide (average particle size =15 nm)

(*8) Aluminum stearate-treated titanium dioxide (average particle size =30 to 35 nm)

As shown in Table 3, the improvements in UV protection ability (boosting effect) by unifying the surface-treated layers of the particulate titanium dioxide and the extender pigment into a metal soap treatment were confirmed in all of the measured Examples 1 to 3. In addition, unifying the surface treatments of the extender pigment, which is a dispersion medium, and the particulate titanium dioxide, which is a disperse phase, into a metal soap treatment reduced the squeaking feeling.

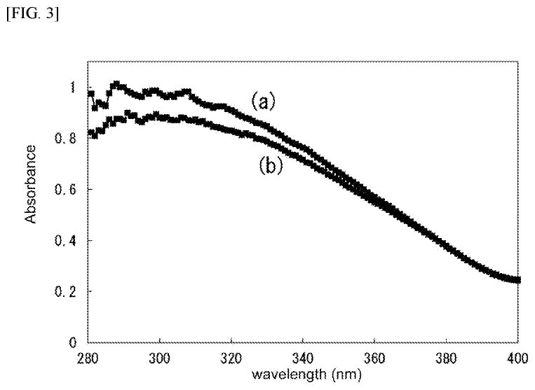

The metal soap-treated titanium dioxide in Example 1 and Comparative Example 1 was replaced with the same amount of another metal soap-treated titanium dioxide (Magnesium isostearate-treated titanium dioxide; average particle size=15 nm or less) to be “Example 4” and “Comparative Example 2”, respectively, and subjected to a measurement of absorbance in the UV wavelength region. The obtained absorbance curves are shown in FIG. 3.

As is clear from FIG. 3, it was confirmed that, even when the surface treatment agent of titanium dioxide was changed from aluminum stearate to magnesium isostearate, the UV protection ability was improved when the metal soap-treated titanium dioxide was dispersed into the metal soap-treated extender pigment (a: Example 4) compared to when the metal soap-treated titanium dioxide was dispersed into the silicone-treated extender pigment (b: Comparative Example 2).

Next, the change in UV protection effect was measured in the composition comprising titanium dioxide surface-treated with metal soap (metal soap-treated titanium dioxide) and the extender pigment surface-treated with metal soap (metal soap-treated extender pigment) (“Sample A1” in Table 1; hereinafter referred to as “Example X”), using compositions (Examples 5 to 7) in which a portion of the metal soap-treated talc was replaced with silica (spherical silica). Furthermore, the change in UV protection effect was measured by preparing a composition (Comparative Example 3) having 30% by mass of metal soap-treated titanium dioxide and the remaining amount of untreated mica, and using a composition (Example 8) in which a portion (10% by mass) of the untreated mica was replaced with metal soap-treated mica in the composition of Comparative Example 3, and compositions (Examples 9 to 11) in which a portion of the untreated mica was replaced with silica (spherical silica) in the composition of Example 8.

Specifically, powder cosmetics were prepared according to the formulations listed in Tables 4 and 5 below. In the same manner as Test Examples 1 and 2described above, 10 mg of the cosmetic of each example was applied to a PMMA plate and subjected to a measurement of absorbance to light in the UV wavelength region of 280 to 400 nm, and then the absorbance in the UV wavelength region was integrated.

In Table 4, the integral value of the absorbance curve obtained from the composition (cosmetic) of “Example X” was taken as the “reference integral value”, and each integral value of the absorbance curves obtained from the compositions (cosmetics) of Examples 5 to 7 was taken as the “measured integral value”, and the value (boost ratio) calculated by the expression described in paragraph 0046 was calculated.

In Table 5, the integral value of the absorbance curve obtained from the composition (cosmetic) of “Comparative Example 3” in which “metal soap-treated titanium dioxide” was dispersed in “untreated mica” was taken as the “reference integral value”, and each integral value of the absorbance curves obtained from the compositions (cosmetics) of Examples 8 to 11 was taken as the “measured integral value”, and the value (boost ratio) calculated by the expression described in paragraph 0046 was calculated.

TABLE 4

| | Example X | Example 5 | Example 6 | Example 7 |
|---|---|---|---|---|
| Metal soap-treated talc (*2) | Remaining | Remaining | Remaining | Remaining |
| | amount | amount | amount | amount |
| Silicone-treated talc (*3) | — | — | — | — |
| Metal soap-treated titanium | 15 | 15 | 15 | 15 |
| dioxide (*1) | | | | |
| Spherical silica powder | — | 10 | 20 | 30 |
| (specific surface area: 600 to | | | | |
| 800 m²/g) | | | | |
| Total | 100 | 100 | 100 | 100 |
| UV protection ability (boost ratio) | Reference | 115 | 135 | 142 |

TABLE 5

| | Comparative | | | | |
|---|---|---|---|---|---|
| | Example 3 | Example 8 | Example 9 | Example 10 | Example 11 |
| Untreated mica | Remaining | Remaining | Remaining | Remaining | Remaining |
| | amount | amount | amount | amount | amount |
| Metal soap-treated mica (*5) | — | 10 | 10 | 10 | 10 |
| Metal soap-treated titanium | 30 | 30 | 30 | 30 | 30 |
| dioxide (*1) | | | | | |
| Spherical silica powder | — | — | 10 | 20 | 30 |
| (specific surface area: 600 to | | | | | |
| 800 m²/g) | | | | | |
| Total | 100 | 100 | 100 | 100 | 100 |
| UV protection ability (boost | Reference | 104 | 108 | 110 | 114 |
| ratio) | | | | | |

As shown in Tables 4 and 5, formulating (c) silica (spherical silica) further improved the UV protection ability. It was confirmed that the boosting effect of the UV protection ability increases along with the increase of the amount of silica

Furthermore, formulating the spherical silica further improved texture (absence of squeakiness).

Formulation Examples of the powder cosmetic (loose powder and pressed powder) according to the present invention are described below. In each formulation, texture without squeakiness and high UV protection effect were obtained.

### Formulation Example 1 (Loose Powder)

TABLE 6

| Component | Amount (% by mass) |
|---|---|
| Mica | Remaining amount |
| Metal soap-treated mica | 10 |
| Boron nitride | 10 |
| Amino acid powder (lauroyl lysine) | 2 |
| Starch powder | 2 |
| Metal soap (Ca myristate) | 6 |
| Barium sulfate | 1 |
| Porous silica | 10 |
| Non-porous silica | 15 |
| Metal soap-treated titanium dioxide | 25 |
| Zinc oxide | 7 |
| Preservative | Appropriate amount |
| Antioxidant | Appropriate amount |
| Coloring material | 1.5 |
| Pearly pigment | 0.2 |
| Perfume | Appropriate amount |
| Total | 100 |

### Formulation Example 2 (Loose Powder)

TABLE 7

| Component | Amount (% by mass) |
|---|---|
| Mica | Remaining amount |
| Metal soap-treated mica | 10 |
| Barium sulfate | 1 |
| Porous silica | 30 |
| Non-porous silica | 10 |
| Metal soap-treated titanium dioxide | 25 |
| Zinc oxide | 7 |
| Preservative | Appropriate amount |
| Antioxidant | Appropriate amount |
| Coloring material | 1.5 |
| Pearly pigment | 10 |
| Perfume | Appropriate amount |
| Total | 100 |

### Formulation Example 3 (Pressed Powder)

TABLE 8

| Component | Amount (% by mass) |
|---|---|
| Mica | Remaining amount |
| Metal soap-treated mica | 30 |
| Metal soap (Ca myristate) | 5 |
| Porous silica | 10 |
| Non-porous silica | 3 |
| Metal soap-treated titanium dioxide | 15 |
| Zinc oxide | 3 |
| Petrolatum | 1 |
| Sorbitan sesquiisostearate | 1 |
| Triisostearin | 1 |
| Triethylhexanoin | 1 |
| Preservative | Appropriate amount |
| Antioxidant | Appropriate amount |
| Coloring material | 8 |
| Pearly pigment | 8 |
| Perfume | Appropriate amount |
| Total | 100 |

1. A powder cosmetic, comprising:
(a) a metal oxide powder consisting of at least one selected from titanium dioxide, zinc oxide, and cerium oxide; and
(b) an extender pigment,
wherein each of the (a) metal oxide powder and the (b) extender pigment is a powder surface-treated with metal soap, and
wherein the amount of the (a) metal oxide powder is 10% by mass or more with respect to the total amount of the cosmetic.

2. The powder cosmetic according to claim 1, wherein an average particle size of the (a) metal oxide powder is 100 nm or less.

3. The powder cosmetic according to claim 1, wherein the metal soap comprises aluminum stearate.

4. The powder cosmetic according to claim 1, further comprising (c) silica, provided that the (c) silica is other than those satisfying the (b).

5. The powder cosmetic according to claim 4, wherein a specific surface area of the (c) silica is 160 m²/g or more.

6. The powder cosmetic according to claim 1, wherein the amount of an oil is 10% by mass or less with respect to the total amount of the cosmetic.
